(19) United States
(12) Patent Application Publication
(10) Pub. No.: US 2012/0108985 A1
(43) Pub. Date: May 3, 2012

(54) CUFFLESS BLOOD PRESSURE MONITOR

(75) Inventors: LIANG-YU SHYU, NEW TAIPEI CITY (TW); YAO Lin KAO, KAOHSIUNG CITY (TW); WEN Ya TSAI, HUALIEN COUNTY (TW); WEI-CHIH HU, TAOYUAN COUNTY (TW); CHING-SUNG WENG, TAOYUAN COUNTY (TW)

(73) Assignee: CHUNG YUAN CHRISTIAN UNIVERSITY, CHUNG LI (TW)

(21) Appl. No.: 13/242,368

(22) Filed: Sep. 23, 2011

(30) Foreign Application Priority Data
Oct. 27, 2010 (TW) 099136821

(51) Int. Cl. A61B 5/021

(52) U.S. Cl. 600/485

(57) ABSTRACT

A cuffless blood pressure monitor is revealed. The cuffless blood pressure monitor includes a pressure detection module, a signal processing module and a display module. The pressure detection module detects blood pressure to generate a blood pressure pulse signal. The signal processing module processes the blood pressure pulse signal to generate a measurement result that is displayed by the display module. The cuffless blood pressure monitor measures continuous blood pressure pulse signals, processes the blood pressure pulse signals by the signal processing module, and calculates the measurement result for real-time measurement of blood pressure. A measurement point on the user is pressed by a soft pressure-transferring medium so that the user won't feel uncomfortable. Moreover, the cuffless blood pressure monitor is compact and portable.

### BACKGROUND OF THE INVENTION

1. Fields of the Invention

The present invention relates to a blood pressure monitor, especially to a cuffless blood pressure monitor.

2. Descriptions of Related Art

For the developed countries, the common diseases have changed from infectious diseases to chronic diseases. Hypertension, diabetes, and stoke are the most common chronic diseases in the world. Especially, patients with high blood pressure, they may have other health problems. Just like electrocardiogram (ECG), blood pressure (BP) is used as a quantitative index for evaluating the health condition of the heart. Many physiological responses have effects on and cause changes of the blood pressure. Thus blood pressure measurement is essential no matter in clinical monitoring or routine check.

Various blood pressure monitoring methods and devises is available now. Most of the blood pressure monitoring devices fall into two categories-invasive type and noninvasive type.

In clinical use, a catheter is inserted into a radial artery or left ventricle through peripheral vessels for invasive blood pressure measurement or cardiac monitoring. This method is with high accuracy. However, the invasive method is rarely used in routine testing. The non-invasive method such as auscultator method, oscillometric method, etc. is used for routine blood pressure measurement.

The non-invasive blood pressure monitor includes an air pump and a cuff so as to increase the pressure around 200 mmHg. Thus, the main disadvantage of the non-invasive blood pressure monitor available on the market now is the discomfort of the user caused by the high cuff pressure during the measurement. However, not all patients can tolerate this and some may feel pain or discomfort. Moreover, the size of the blood pressure monitor is very large so that it's not portable.

In order to solve the above problems, a novel blood pressure monitor that overcomes the shortcomings of automatic blood pressure monitors is invented. The limitations include: the measurement of continuous blood pressure signal, difficulty in carrying out the automatic blood pressure monitor due to the large size and discomfort caused by the pressure from the inflated cuff that is required for automatic blood pressure monitoring.

### SUMMARY OF THE INVENTION

The primary object of the present invention is to provide a cuffless blood pressure monitor that measures continuous blood pressure pulse signals, processes the blood pressure pulse signals by the signal processing module, and calculates the measurement result for real-time measurement of blood pressure.

The another object of the present invention is to provide a cuffless blood pressure monitor in which a soft, pressure-transferring medium substitute the inflated cuffs of common automatic blood pressure monitors. The user presses the soft, pressure-transferring medium again the point of measurement on the user's hand such that the user will not feel uncomfortable during the measurement.

The further object of the present invention is to provide a cuffless blood pressure monitor that does not require an inflated cuff as in an automatic blood pressure monitor. Thus the cuffless blood pressure monitor is small and truly portable.

The further object of the present invention is to provide a cuffless blood pressure monitor that processes and calculates the measured blood pressure pulse signals though a special signal processing method so as to obtain an accurate measurement result.

In order to achieve the above objects, a cuffless blood pressure monitor of the present invention is composed of a pressure detection module, a signal processing module and a display module. The pressure detection module consists of a pressure-transferring medium and a pressure sensor. The pressure-transferring medium covers the sensing end of the pressure sensor. The signal processing module is connected to the pressure sensor of the pressure detection module. The pressures sensor senses the blood pressure pulse signal and sends the blood pressure pulse signal to the signal processing module. The signal processing module processes the blood pressure pulse signal to obtain the blood pressure direct current signal and the blood pressure alternating current signal. Then a measurement result is calculated and obtained according to the blood pressure direct current signal and the blood pressure alternating current signal. The display module is connected to the signal processing module so as to display the measurement result calculated by the signal processing module.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a cross sectional view of an embodiment according to the present invention;

FIG. 2 is a block diagram of an embodiment according to the present invention;

FIG. 3 is a block diagram of a signal processing module of an embodiment according to the present invention;

FIG. 4 is a block diagram of another embodiment according to the present invention;

FIG. 5 is a cross sectional view of another embodiment according to the present invention;

FIG. 6 is a cross sectional view of a further embodiment according to the present invention.

### DETAILED DESCRIPTION OF THE PREFERRED EMBODIMENT

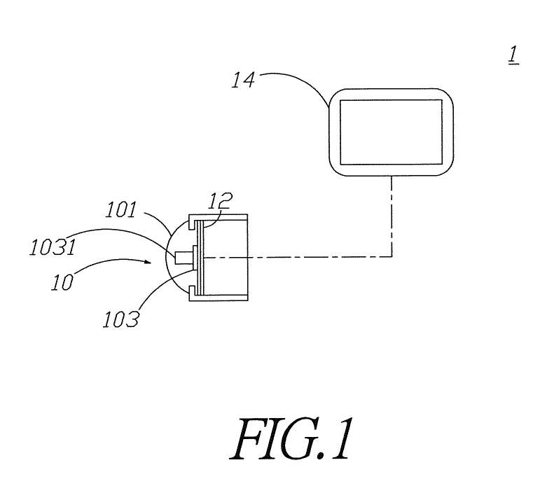

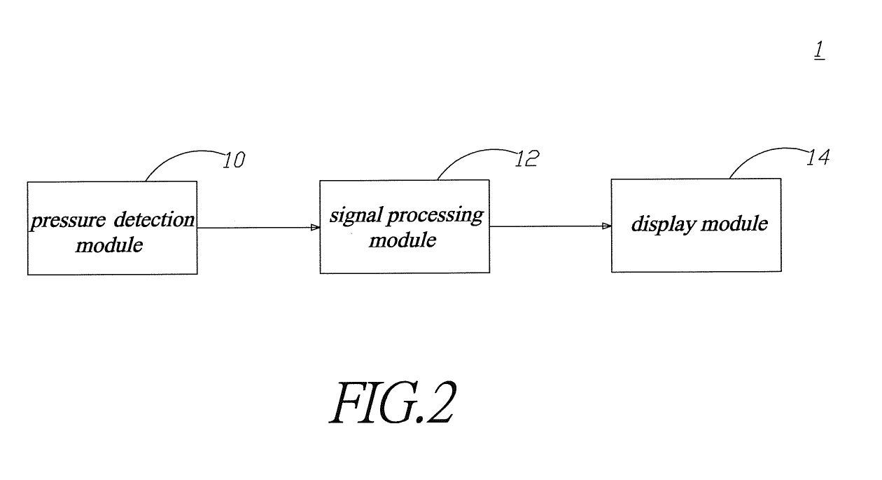

Refer to FIG. 1 and FIG. 2, a cross sectional view and a block diagram of an embodiment are disclosed. As shown in figure, a cuffless blood pressure monitor 1 of the present invention is a non-invasive blood pressure monitor. All the non-invasive blood pressure monitors currently available include a cuff. The inflation of the cuff applies a pressure to an artery of an user. However, the applied pressure will make the user feel uncomfortable. Thus the present invention provides a cuffless blood pressure monitor 1.

The cuffless blood pressure monitor 1 includes a pressure detection module 10, a signal processing module 12 and a display module 14. The pressure detection module 10 consists of a pressure-transferring medium 101, and a pressure sensor 103. The pressure-transferring medium 101 covers a sensing end 1031 of the pressure sensor 103. The pressure-transferring medium 101 has properties of air tightness and flexibility so as to transmit the pressure difference between the detected area and the pressure-transferring medium 101 to the pressure sensor 103. The pressure sensor 103 senses the blood pressure pulse signal according to the pressure change between the detected area and the pressure-transferring medium 101. In this embodiment, the pressure-transferring medium 101 is made from silicone. The pressure-transferring medium 101 can also be made from other soft materials.

The signal processing module 12 is connected to the pressure sensor 103 of the pressure detection module 10. The blood pressure pulse signal sensed by the pressure sensor 103 is sent to the signal processing module 12. The signal processing module 12 processes the blood pressure pulse signal to obtain a blood pressure direct current (DC) signal and a blood pressure alternating current (AC) signal. Then the signal processing module 12 calculates and obtains a measurement result according to the blood pressure DC signal and the blood pressure AC signal. The measurement result includes a diastolic pressure, a systolic pressure, and a mean blood pressure.

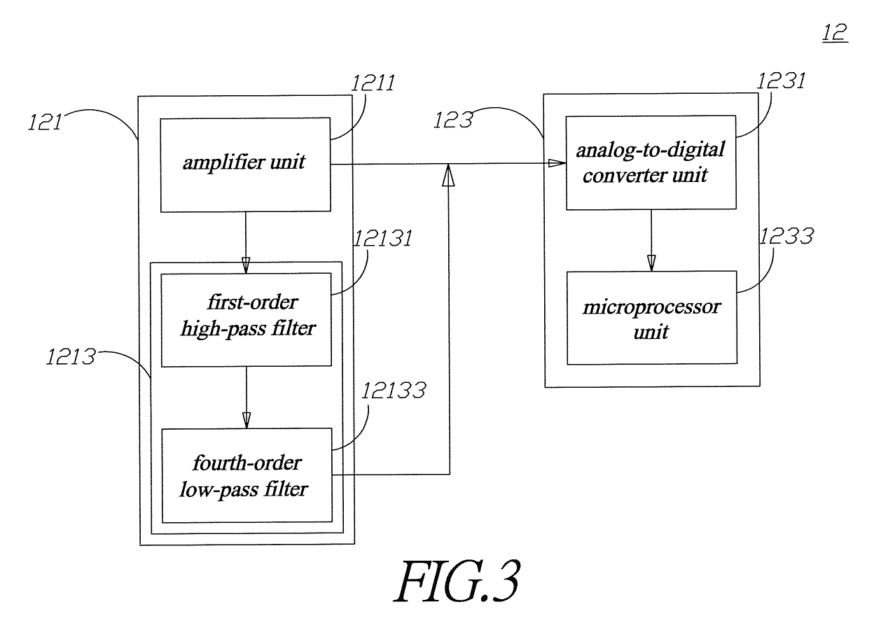

Refer to FIG. 3, an embodiment of the signal processing module 12 is revealed. As shown in the figure, the embodiment of the signal processing module 12 consists of an analog signal processing circuit 121 and a digital signal processing circuit 123. The blood pressure pulse signal from the pressure sensor 103 of the pressure detection module 10 which is an analog signal is transmitted to the analog signal processing circuit 121 of the signal processing module 12 firstly. The analog signal processing circuit 121 is composed of an amplifier unit 1211 and a filter unit 1213. The blood pressure pulse signal is amplified by the amplifier unit 1211 and then is divided into the blood pressure DC signal and the blood pressure AC signal. The filter unit 1213 receives the blood pressure AC signal. Finally at least one high frequency noise or low frequency noise in the blood pressure AC signal is removed by the filter unit 1213. In this embodiment, a differential amplifier is used as the amplifier unit 1211 while the filter unit 1213 includes a first-order high-pass filter 12131 and a fourth-order low-pass filter 12133.

After being processed by the analog signal processing circuit 121, the blood pressure DC signal and the blood pressure AC signal are sent to the digital signal processing circuit 123. The digital signal processing circuit 123 includes an analog-to-digital converter unit 1231 and a microprocessor unit 1233. The analog-to-digital converter unit 1231 is used to receive and digitalize both the blood pressure DC signal and the blood pressure AC signal.

The pressure applied by the embodiment of the cuffless blood pressure monitor 1 is controlled by manual operation of the user. When the force applied is not even, the amplitude of the blood pressure pulse signal generated by the pressure sensor 103 will not increased linearly. Thus a special signal processing of the pulse signal by the microprocessor unit 1233 is required. At first, the microprocessor unit 1233 retrieves multiple amplitude values of the digitalized blood pressure AC signal and checks whether these amplitude values exceed a threshold value. Then find out the corresponding blood pressure values in the digitalized blood pressure DC signal for blood pressure AC signals that exceed the threshold value. Next rearrange the blood pressure values of the blood pressure DC signal from largest to smallest so that a curve of the rearranged blood pressure DC signal is a monotonously decline curve. Thus the so-called rearranged blood pressure DC signal is obtained.

Next the microprocessor unit 1233 rearranges the digitalized blood pressure AC signal according to the order of the blood pressure values of the rearranged blood pressure DC signal. Then the microprocessor unit 1233 finds out the maximum value of the amplitude of the rearranged blood pressure AC signal so as to get the blood pressure value corresponding to the maximum value of the amplitude. Moreover, the measurement result such as the systolic pressure, the diastolic pressure and the mean blood pressure is calculated and obtained according to the characteristic ratio between the maximum amplitude and blood pressure amplitude at systolic pressure and the diastolic pressure.

Refer back to FIG. 2, the display module 14 is connected to the signal processing module 12. After being calculated by the signal processing module 12, the measurement result is sent to the display module 14 to be displayed. In this embodiment, the display module 14 can be a liquid crystal display (LCD), a plasma display panel (PDP), or Organic light emitting diodes (OLED) display.

Refer back to FIG. 1, both the pressure detection module 10 and the signal processing module 12 of the embodiment of the cuffless blood pressure monitor 1 are mounted inside a main body 16. The display module 14 is arranged outside the main body 16 and is connected to the signal processing module 12.

To use the invention, the user sits upright properly, feels the pulse and finds out the place with the maximum pulse at the radial artery near the left wrist as a measurement point. Then place the surface of the cuffless blood pressure monitor 1 with the pressure-transferring medium 101 on the measurement point. The user himself/herself holds the main body 16 of the cuffless blood pressure monitor 1 and applies pressure to the measurement point. The pressure sensor 103 detects the pressure applied by the user and sends the press signal to the signal processing module 12. According to the press signal, the signal processing module 12 gets the pressure on the measurement point applied by the user. The signal processing module 12 sends the pressure value to the display module 14 and the pressure value is displayed as a pressure bar by the display module 14. The user continues applying force to the measurement point until the pressure bar shown on the display module 14 start to blink and the measurement is completed.

During the measurement, the pressure sensor 103 of the pressure detection module 10 senses the blood pressure signal and sends the blood pressure signal to the signal processing module 12. The analog signal processing circuit 121 of the signal processing module 12 receives the blood pressure signal. The blood pressure signal is firstly amplified by the amplifier unit 1211 and then is divided into the blood pressure DC signal and the blood pressure AC signal. The blood pressure DC signal is the signal of the pressure applied while the blood pressure AC signal represents the pulse pressure signal. Next the filter unit 1213 removes the high frequency noise or low frequency noise from the blood pressure AC signal. Finally, the blood pressure DC signal and the blood pressure AC signal are transmitted to the digital signal processing circuit 123.

In the digital signal processing circuit 123, the analog-to-digital converter unit 1231 digitalizes both the blood pressure DC signal and the blood pressure AC signal. Then the microprocessor unit 1233 processes the digitalized blood pressure DC signal and the digitalized blood pressure AC signal. And the measurement result is calculated and obtained according to the blood pressure DC signal and the blood pressure AC signal. How the microprocessor unit 1233 processes the digitalized blood pressure DC signal and the digitalized blood pressure AC signal has been described in the above embodiment. At last, the digital signal processing circuit 123 sends the measurement result to the display module 14 for showing the measurement result.

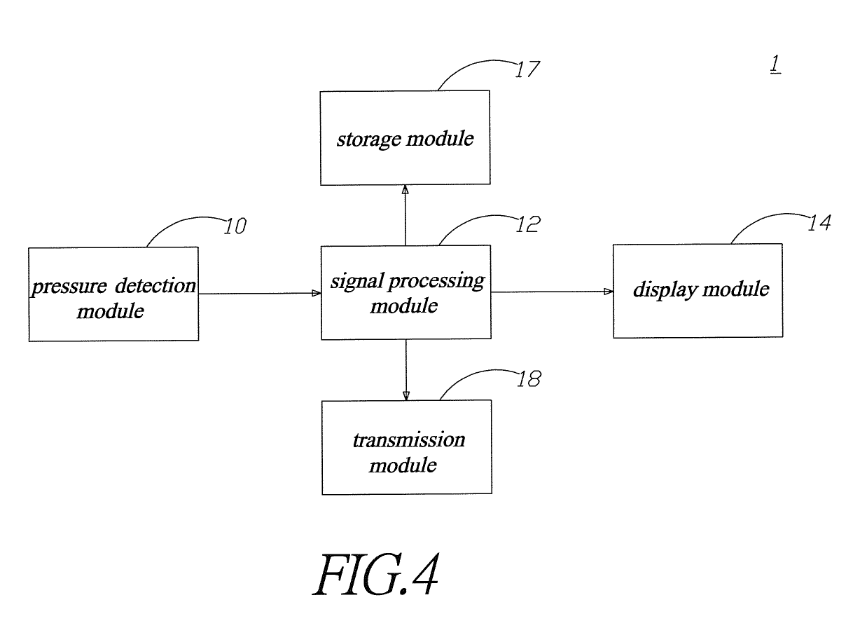

Refer to FIG. 4, a block diagram of another embodiment according to the present invention is revealed. As shown in figure, the difference between this embodiment and the above embodiment of a cuffless blood pressure monitor 1 is in that: this embodiment of the cuffless blood pressure monitor 1 further includes a storage module 17 and a transmission module 18. The storage module 17 is connected to the signal processing module 12 so as to store the blood pressure DC signal, the blood pressure AC signal and the measurement result. In this embodiment, the storage module 17 is a flash memory.

The transmission module 18 is connected to the signal processing module 12 and is used for sending the blood pressure DC signal, the blood pressure AC signal and the measurement result to a computer. In this embodiment, the transmission module 18 is a universal serial bus (USB) device.

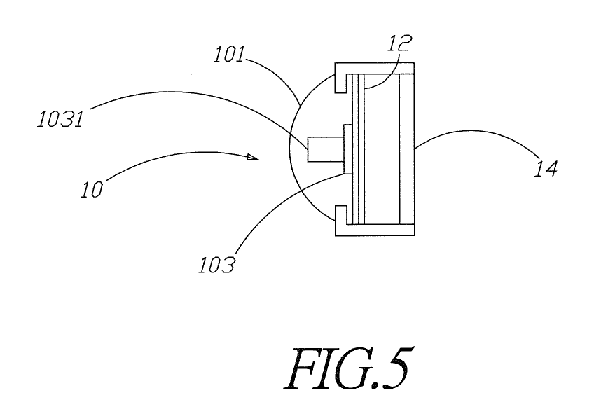

Refer to FIG. 5, a cross sectional view of another embodiment according to the present invention is revealed. As shown in figure, the different between this embodiment and the above embodiment is in that the display module 14 of this embodiment is arranged at the main body 16. Thus users can carry the cuffless blood pressure monitor by themselves and use the device conveniently, anytime when it is needed.

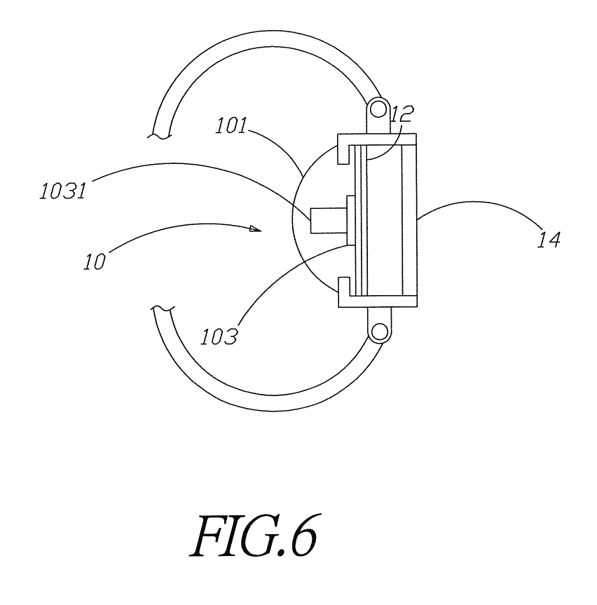

Refer to FIG. 6, a cross sectional view of a further embodiment according to the present invention is disclosed. This embodiment combines the cuffless blood pressure monitor 1 shown in FIG. 5 with a watch. The user wears the watch with the cuffless blood pressure monitor 1 on the wrist directly. While measuring the blood pressure, the user sets the pressure detection module 10 of the cuffless blood pressure monitor 1 on the radial artery of the user's wrist. Then the user presses the surface of the watch and applies pressures to the measurement point for blood pressure measurement. The cuffless blood pressure monitor 1 shown in FIG. 5 can also be combined with a mobile phone. The cuffless blood pressure monitor 1 is arranged at one of the four corners of the mobile phone so that the users can apply pressure to the measurement point easier.

In summary, the cuffless blood pressure monitor 1 of the present invention mainly overcomes shortcomings of automatic blood pressure monitors available on the market now. The limitation includes cannot obtain the continuous blood pressure signal, difficulty in carrying the automatic blood pressure monitor due to the large size and discomfort caused by the pressure from the inflated cuff. The cuffless blood pressure monitor 1 of the present invention can measure the continuous blood pressure pulse signal, processes the blood pressure pulse signal by the signal processing module and calculates the measurement result. Thus the blood pressure is measured in real time.

Instead of the inflating cuff of common automatic blood pressure monitors, the present invention uses soft pressure-transferring medium to apply pressure to the measurement point on user's arm. Thus the user will not feel uncomfortable during blood pressure measurement.

Moreover, without the cuff of common automatic blood pressure monitors, the cuffless blood pressure monitor of the present invention is compact and portable by virtue of the smaller size.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed:

1. A cuffless blood pressure monitor comprising:
a pressure detection module having a pressure-transferring medium and a pressure sensor and the pressure-transferring medium covering a sensing end of the pressure sensor;
a signal processing module that is connected to the pressure sensor of the pressure detection module; wherein the pressure sensor senses and sends a blood pressure pulse signal to the signal processing module to be processed so as to get a blood pressure direct current signal and a blood pressure alternating current signal and further calculate and obtain a measurement result according to the blood pressure direct current signal and the blood pressure alternating current signal; and
a display module that is connected to the signal processing module so as to display the measurement result calculated by the signal processing module.

2. The device as claimed in claim 1, wherein the pressure-transferring medium is made from silicone or other soft material.

3. The device as claimed in claim 1, wherein the measurement result includes a diastolic pressure, a systolic pressure, and a mean blood pressure.

4. The device as claimed in claim 1, wherein the signal processing module includes:
an analog signal processing circuit that receives the blood pressure pulse signal from the pressure sensor so as to get the blood pressure direct current signal and the blood pressure alternating current signal; and
a digital signal processing circuit that receives, digitalizes both the blood pressure direct current signal and the blood pressure alternating current signal, and calculates the measurement result according to the digitalized blood pressure direct current signal and the digitalized blood pressure alternating current signal.

5. The device as claimed in claim 4, wherein the analog signal processing circuit includes
an amplifier unit that amplifies the blood pressure signal and then the blood pressure pulse signal is divided into the blood pressure direct current signal and the blood pressure alternating current signal; and
a filter unit that receives the blood pressure alternating current signal and removes at least one noise from the blood pressure alternating current signal.

6. The device as claimed in claim 5, wherein the amplifier unit is a differential amplifier.

7. The device as claimed in claim 5, wherein the filter unit includes a high-pass filter and a low-pass filter.

8. The device as claimed in claim 4, wherein the digital signal processing circuit includes:
an analog-to-digital converter unit that receives both the blood pressure direct current signal and the blood pressure alternating current signal from the analog signal processing circuit, and digitalizes both the blood pressure direct current signal and the blood pressure alternating current signal; and
a microprocessor unit that receives both the digitalized blood pressure direct current signal and the digitalized blood pressure alternating current signal, retrieves multiple amplitude values of the digitalized blood pressure AC signal and checks whether these amplitude values exceed a threshold value; then find out the corresponding blood pressure DC signal for those blood pressure AC signals that exceed the threshold value; next rearrange the blood pressure values of the blood pressure DC signal from largest to smallest so that a curve of the rearranged blood pressure DC signal is a monotonously decline curve; the so-called rearranged blood pressure DC signal is obtained; wherein the microprocessor unit rearranges the digitalized blood pressure alternating current signal according to the order of the blood pressure values of the rearranged blood pressure direct current signal; then the microprocessor unit finds out the maximum value of an amplitude of the rearranged blood pressure alternating current signal so as to get a blood pressure value corresponding to the maximum value of the amplitude and calculate the measurement result according to the characteristic ratio between the maximum amplitude and blood pressure amplitude at systolic pressure and the diastolic pressure

9. The device as claimed in claim 1, wherein the cuffless blood pressure monitor further includes: a storage module that is connected to the signal processing module so as to store the blood pressure direct current signal, the blood pressure alternating current signal and the measurement result.

10. The device as claimed in claim 9, wherein the storage module is a flash memory.

11. The device as claimed in claim 1, wherein the cuffless blood pressure monitor further includes: a transmission module that is connected to the signal processing module and is used for sending the blood pressure direct current signal, the blood pressure alternating current signal and the measurement result to a computer.

12. The device as claimed in claim 11, wherein the transmission module is a universal serial bus (USB) device.

13. The device as claimed in claim 1, wherein the pressure sensor senses the press signal and sends the press signal to the signal processing module; the signal processing module gets a pressure value according to the press signal and sends the pressure value to the display module; the pressure value is displayed by the display module.
